(19) United States
(12) Patent Application Publication
(10) Pub. No.: US 2009/0131827 A1
(43) Pub. Date: May 21, 2009

(54) APPARATUS AND METHODS FOR TISSUE DISRUPTION

(75) Inventors: Michael D. CROCKER, Half Moon Bay, CA (US); Thomas E. Brockman, Hayward, CA (US)

Correspondence Address: LEVINE BAGADE HAN LLP, 2483 EAST BAYSHORE ROAD, SUITE 100, PALO ALTO, CA, 94303, US

(73) Assignee: StemCor Systems, Inc., Menlo Park, CA (US)

(21) Appl. No.: 11/944,210

(22) Filed: Nov. 21, 2007

(51) Int. Cl. A61B 10/02

(52) U.S. Cl. 600/571; 604/22

(57) ABSTRACT

Apparatus and methods for tissue disruption are disclosed where a tissue disruptor may have various configurations extending from the distal end of a flexible aspiration cannula. The devices can have aspiration and/or irrigation systems configured to provide aspiration pressure and/or irrigate with fluid at the distal end of the cannula. The cannula can be configured to rotate or disrupt the matrix of bone marrow and extract the marrow in vivo through a single opening. The cannula shaft itself may be fabricated utilizing multiple layers of material such that the cannula is flexible yet sufficiently stiff to transmit a torque therealong.

### FIELD OF THE INVENTION

The present invention relates to devices and methods for extraction of tissue from an enclosed body cavity. More particularly, the present invention relates to devices and methods for harvesting bone marrow through a single entry port from an enclosed bone cavity.

### BACKGROUND OF THE INVENTION

Bone Marrow is a rich source of pluripotent hematopoietic stem cells from which red blood cells, white blood cells, and platelets are formed. Bone marrow also contains additional populations of mesenchymal stem cells and other stem and progenitor cells which have the potential to repair and regenerate other tissues.

Since the early 1970's bone marrow and hematopoietic stem cell transplantation has been used to treat patients with a wide variety of disorders, including but not limited to cancer, genetic and autoimmune diseases. Currently over 60,000 transplants for a variety of indications are performed worldwide each year.

In autologous transplants, the patient has their own bone marrow collected prior to receiving high dose chemotherapy. Following high dose, myeloablative chemotherapy, which kills the majority of the patients' marrow stem cells, the stored autologous marrow or hematopoietic stem cells purified or enriched from the marrow are infused, and serves to improve the patient's hematolymphoid system.

In allogeneic transplants bone marrow, or other sources of hematopoietic stem cells derived from a full or partially human leukocyte antigen (HLA) matched sibling, parent or unrelated donor is infused into the recipient patient and following engraftment, serves to reconstitute the recipients hematopoietic system with cells derived from the donor.

Following myeloablative or non-myeloablative conditioning of a patient with chemotherapy and/or radiation therapy, the marrow is regenerated through the administration and engraftment of hematopoietic stem cells contained in the donor bone marrow.

In addition to hematopoietic stem cells and hematopoietic progenitors, bone marrow contains mesenchymal and other stem cell populations thought to have the ability to differentiate into muscle, myocardium, vasculature and neural tissues and possibly some organ tissues such as liver and pancreas. Research in preclinical animal studies and clinical trials suggest that bone marrow or some portion of the cells contained within marrow can regenerate tissues other than the hematopoietic system. This includes the ability for cells contained within the marrow to regenerate or facilitate repair of myocardial tissue following a myocardial infarction, and in the setting of congestive heart failure as evident by improved cardiac function and patient survival.

Bone marrow derived stem cells also show evidence for their ability to regenerate damaged liver and hepatic cells and portions of the nervous system including spinal cord. Additional organ systems including kidney and pancreas show benefit from bone marrow derived cells. Use of bone marrow and the stem cells contained within bone marrow may be of increasing clinical utility in the future treatment of patients. Furthermore a patient's own marrow has multiple applications in orthopedic procedures, including but not limited to spinal fusions, treatment of non-union fractures, osteonecrosis, and tissue engineering.

Stem cells utilized in transplantation are usually collected using one of two methods. In a first method known as a bone marrow harvest, bone marrow is directly accessed in and removed from the patient usually by multiple aspirations of marrow from the posterior iliac crest. The bone marrow harvest procedure is often performed in the operating room.

To perform a harvest of 500-1500 milliliters of marrow, multiple separate entries into the marrow cavity are required to in order to remove a sufficient amount of bone marrow. A bone marrow aspiration needle, such as a sharp metal trocar, is placed into the marrow space through the soil tissue and the outer cortex of the iliac crest. The aspiration needle enters less than 2 cm into the marrow cavity. Negative pressure is applied through the hollow harvest needle, usually by the operator pulling on an attached syringe into which 5-10 ml of marrow is aspirated. The needle and syringe are then removed.

After removing the collected marrow, the aspiration needle accesses a separate location on the iliac bone for another aspiration. This method of inserting the needle into the bone, removing the marrow, and removing the needle from the bone is performed on the order of 100-200 separate entries for an average patient to remove a volume of bone marrow required for transplantation.

Each puncture and entry into the marrow cavity accesses only a limited area of the marrow space, and the majority of practitioners only remove 5-10 milliliters of marrow with each marrow penetration. Pulling more marrow from a single marrow entry site otherwise results in a collected sample highly diluted by peripheral blood.

The bone marrow harvest procedure requires general anesthesia because the iliac crest is penetrated 100-300 times with a sharp bone marrow trocar. Local anesthesia is generally not possible given the large surface area and number of bone punctures required.

The donor needs time to recover from general anesthesia, and frequently suffers from days of sore throat, a result of the endotracheal intubation tube placed in the operating room.

Pre-operative preparation, the harvest procedure, recovery from anesthesia, and an overnight observation stay in the hospital following the procedure requires considerable time on behalf of the donor and the physician, and similarly additional expense. The cost of the procedure is often $10,000 to $15,000, which includes costs for operating room time, anesthesia supplies and professional fees, and post-operative care and recovery.

In addition to general operating room staff, the traditional bone marrow harvest procedure requires two transplant physicians. Each physician aspirates marrow from the left or right side of the iliac crest. The procedure itself usually takes approximately one and half hours for each operating physician.

Many donors experience significant pain at the site of the multiple bone punctures which persists for days to weeks.

Traditional bone marrow aspiration incurs a significant degree of contamination with peripheral blood. Peripheral blood contains high numbers of mature T-cells unlike pure bone marrow. T-cells contribute to the clinical phenomenon termed Graft vs. Host Disease (GVHD), in both acute and chronic forms following transplant in which donor T-cells present in the transplant graft react against the recipient (host) tissues. GVHD incurs a high degree of morbidity and mortality in allogeneic transplants recipients.

In a second method to collect stem cells for transplantation, mononuclear cells are removed from the donor's peripheral blood. The peripheral blood contains a fraction of hematopoietic stem cells as well as other populations of cells including high numbers of T-cells. In this procedure peripheral blood stem cells are collected by apheresis following donor treatment with either chemotherapy—usually cyclophosphamide—or with the cytokine Granulocyte Colony Stimulating Factor (GCSF). Treatment with cyclophosphamide or GCSF functions to mobilize and increase the numbers of hematopoietic stem cells circulating in the blood.

This collection method can be slow and time consuming. It requires the donor to first undergo five or more days of daily subcutaneous injections with high doses of the cytokine GCSF prior to the collection. These daily injections can be uncomfortable and painful and bone pain is a common side effect. Peripheral blood stem cells can not be obtained without this seven-plus day lead time.

Each day of apheresis costs approximately $3,000 including but not limited to the cost of the apheresis machine, nursing, disposable supplies and product processing. The patient often has to come back on multiple days in order to obtain an adequate number of stem cells. Costs for the GCSF drug alone approximate $6,000-$10,000 depending upon the weight of the patient.

Given the multiple days required to collect adequate numbers of hematopoietic stem cells, individual bags of peripheral blood product must processed and frozen separately. These bags are then thawed, and given back to the recipient patient at the time of transplant. The volume, and chemicals contained in the product freezing media can cause some complications, such as mild side effects, at the time of infusion.

Accordingly, there is a need for a minimally invasive, less expensive, time-efficient bone marrow harvest procedure with minimal complications which does not require general anesthesia, offers fast recovery time, and does not cause significant pain to the bone marrow donor.

### SUMMARY OF THE INVENTION

Devices and methods for manipulation and extraction of body tissue from an enclosed body cavity (e.g., iliac, femur, humerus, other bone, or combinations thereof) are disclosed. The device can have a hollow introduction or entry cannula that can have a trocar. The introduction cannula and a core element can penetrate body tissue, such as the marrow space contained within the iliac. A flexible aspiration cannula can then be inserted through the introduction cannula into body tissue and can be advanced through the body cavity.

The aspiration cannula can have inlet openings near the distal tip through which tissue is aspirated. At the proximal end of the aspiration cannula a negative pressure (i.e., suction) source can provide controlled negative pressure, for example, to increase the aspiration of tissue through the aspiration cannula into a collection reservoir. The aspiration cannula can be withdrawn and positioned for multiple entries through the same tissue entry point, for example, following different paths through the tissue space for subsequent aspiration of more tissue. The aspiration cannula, for example while moving non-linearly, can access a majority of the bone marrow space through a single point of entry. Suction may be optionally applied to the aspiration cannula while accessing the marrow space to increase the harvest of the bone marrow or other aspiratable substances.

A marrow access site can be the anterior iliac crest access site which can be easy to locate and access on a broad array of patients (from thin to obese) and utilizing this access site can also reduce harvest time. The device and method disclosed herein can also control the directionality of the cannula into the marrow cavity via an access guide such that the device can access a majority of bone marrow space in a single bone or marrow cavity in vivo through a single point of entry. Alternatively, the device and method can access multiple diagnostic samples of bone marrow from disparate sites within a single marrow cavity. The device and method can also have aspiration suction controlled to aspirate bone marrow or fat, for example.

The device can have an elongated cannula having a flexible length, a hollow channel, a cannula first end and a cannula second end. Additionally, the device can include a motor which is rotatably connected to the cannula. The cannula may additionally include a tissue disruptor which is attached to or integral with the cannula, e.g., a looped member having a first end and a second end where the first end can be fixed to the cannula such that the whisk extends from the cannula. The second end can also be fixed to the cannula such that the disruptor is configured in a semi-circular or closed loop configuration.

Turning now to the handle, the handle may be configured to actuate and rotate the aspiration cannula via a motor which is driven by a power supply, e.g., a battery or rechargeable battery, and activated via an actuator control. A mechanical transmission may be coupled to the motor to limit or control the rotational speed of the motor depending upon the actuation of the control to either increase, decrease, or limit the speed at which the motor rotates the aspiration cannula.

The aspiration assembly is removably coupled to the handle and may be secured via a locking mechanism. A plurality of openings may be defined along an aspiration assembly interface along a proximal end of the cannula such that bone marrow and/or other aspirants which are drawn proximally through the cannula may enter the aspiration assembly interface to exit through the openings and into aspirant chamber. As the cannula and aspiration assembly interface are rotated, the bone marrow and/or aspirant drawn through the openings and collected within the chamber may be removed from the assembly via an aspirant port opening.

Turning now to the aspiration cannula, while the cannula may generally be flexible enough to allow for bending or curvature of the shaft when advanced within and/or against the bone cavity interior, the cannula is desirably stiff enough to transmit between 20 to 40 in·oz, and preferably 40 in·oz, of torque to rotate the cannula through the bone marrow. The distal portion of the cannula may comprise a tissue disrupter assembly which may be configured in a number of different variations. One variation is a tissue disruptor, e.g., looped member such as a looped wire, retained within a disrupter tube member, which also defines one or more aspiration ports proximal to the disruptor along a side surface of the disruptor tube member. A proximal portion of the tube member may be secured via a crimped member or swage tube disposed over and securing both the cannula shaft and tube member.

Although the proximal portion of the cannula may generally be stiffer relative to the distal portion, the aspiration lumen defined through the length of cannula may remain relatively constant. For instance, the internal diameter of the cannula may be based upon the standard dimensions of a 12 gauge needle. Multiple layers of material may be overlaid to create the desired stiffness along the proximal portion of the shaft.

Turning now to additional variations for the tissue disrupter, aspiration openings may be defined along a side surface of the tube or along an outer side surface of the aspiration cannula to prevent clogging of the openings by bone marrow or other aspirants during an aspiration procedure. In yet another variation of the tissue disruptor a unitary disruptor tip which may be swaged or otherwise attached to the distal end of a cannula shaft. A unitary tissue disrupter may generally comprise a curved or semicircular disruptor member which extends distally from the tubular member to form an opening. One or more aspiration openings may be defined along the tubular member proximal to the disrupter member such that the aspiration openings are in communication with the lumen defined through the tubular member. The portion of the tubular member proximal to the disruptor member may be occluded such that the only aspiration openings are located along the side surfaces of the tubular member to provide for aspiration therethrough. Such a unitary tissue disruptor may be fabricated as a single and integral unit, e.g., from stainless steel or any other suitable material.

When utilizing the devices to aspirate along a path through the bone marrow within the iliac, a void or channel may be created (at least temporarily) within the bone marrow where the aspirated tissue has been removed. If the aspiration cannula is then withdrawn, repositioned, and reintroduced into the bone cavity along a second path which is adjacent to the first aspirated channel, then the aspiration cannula may inadvertently cross one or more times into the emptied first aspirated channel. To inhibit or prevent this from occurring, a space-occupying member may be inserted through the puncture opening and into the first aspirated channel to temporarily occupy the emptied volume. The space-occupying member may have a length which approximates that of the aspiration cannula such that most, if not all, of the empty space within the aspirated channel is occupied.

### BRIEF DESCRIPTION OF THE DRAWINGS

FIG. 1 illustrates an exploded, partially schematic, view of a variation of the device for tissue disruption and aspiration.

FIG. 2 illustrates an assembled, partially schematic view of another variation of the device for tissue disruption and aspiration.

FIG. 3 illustrates an exploded, partially schematic, view of another variation of the device for tissue disruption and aspiration.

FIG. 4 illustrates an assembled, partially schematic view of another variation of the device for tissue disruption and aspiration.

FIGS. 5A to 5D illustrate one method for accessing and harvesting bone marrow with a flexible aspiration cannula through a single entry port within an iliac crest.

FIG. 6 shows a cross-sectional perspective view of one variation of a handle.

FIGS. 7A to 7C show assembly and detail cross-sectional side views, respectively, of a variation of an aspiration cannula.

FIGS. 8A to 8D illustrate assembly and cross-sectional end views of the cannula shaft along a proximal, transitional, and distal portion of the shaft, respectively showing the multiple layers.

FIG. 9 illustrates a cross-sectional side view of the transitional portion of the cannula shaft.

FIG. 10 shows another variation of a tissue disruptor assembly having a tapered tissue disruptor member.

FIG. 11 shows yet another variation of a tissue disruptor assembly having an occluded distal end.

FIGS. 12A to 12D show side, cross-sectional side, and end views, respectively, of another variation of a tissue disruptor which is made of a unitary construction.

FIG. 13 shows a perspective view of the tissue disruptor of FIG. 12A.

FIG. 14 illustrates an example of inserting a space-occupying member into an empty aspirated channel to inhibit or prevent the aspiration cannula from crossing into the void when aspirating bone marrow along an adjacent path.

### DETAILED DESCRIPTION OF THE INVENTION

A tissue disruption and aspiration device having a flexible elongate shaft or cannula which is rotatable about its longitudinal axis may be introduced into a body cavity, e.g., the marrow cavity of a bone such as the iliac, through a single puncture opening. The cannula may be advanced through the cavity along various paths to aspirate the surrounding bone marrow into and through the cannula. The tissue disruptor end effector located at the distal end of the cannula may be configured to rotate about the longitudinal axis of the end effector and agitate or disrupt the contacted tissue from its surrounding tissue matrix to thus facilitate aspiration of the bone marrow. Although the tissue disruptor end effector is configured to disrupt or agitate the bone marrow, it is further configured to inhibit or prevent the end effector from puncturing into or out through the surrounding bone cavity.

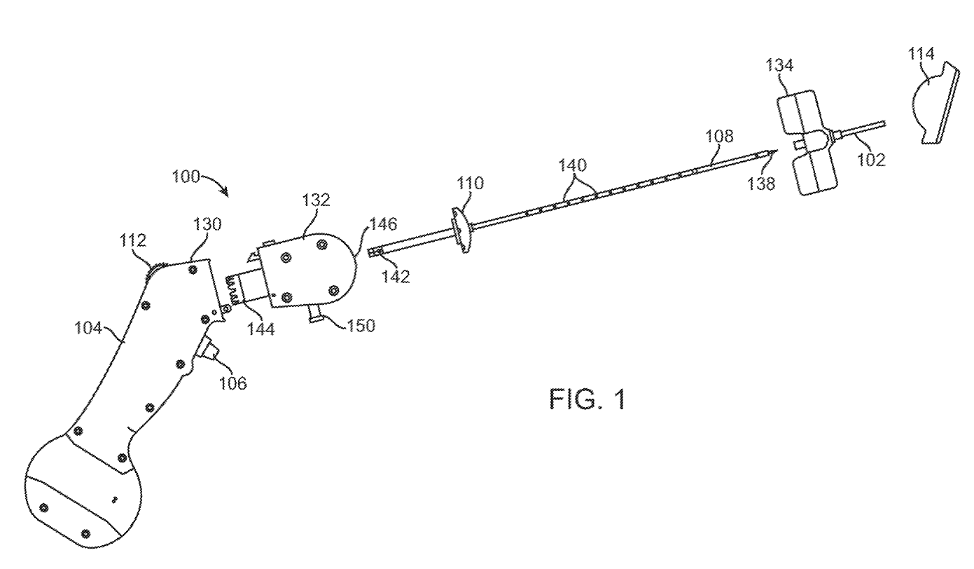

Turning now to FIG. 1, an assembly view is illustrated of a variation of a tissue disruption and aspiration device 100 that can aspirate and collect body tissue from within an enclosed body space in vivo or in vitro (also referred to as “aspiration device”). The aspiration device may generally comprise a drill assembly 130 having a handle 104, a connector and aspiration assembly 132, an aspiration cannula 108, an access trocar 134, and one or more fluid circuits 136.

The aspiration cannula 108 can be removably coupled to the aspiration assembly 132 and/or drill 130 for ease of manipulation and operation such that the aspiration cannula 108 is in mechanical communication with the drill 130. The aspiration cannula 108 may be configured to be flexible and may also include indentations, ridges, rings, or combinations thereof, for example to alter the flexibility of the aspiration cannula 108 along the entire length or a portion of the length of the aspiration cannula 108. Moreover, one or more visualization markers 140 may be defined along a portion or an entire length of the outer surface of aspiration cannula 108 at regular intervals and/or at preset distances to provide a visual indication to the user of a depth of aspiration cannula 108 within the body cavity. Markers 140 may simply comprise gradations or markings and may be also optionally radio-opaque and/or echogenic.

The aspiration cannula 108 may further include a rotational interface 142 configured to rotationally attach or couple to the aspiration assembly 132 and/or the drill 130 through cannula port 146 for transmitting the rotational torque from the drill 130 to the cannula 108. The aspiration cannula 108 can further include a guard and/or a squash plate 110 to prevent over-insertion of the aspiration cannula into the connector 132 and/or the drill 130. The guard 110 can be non-rotationally attached to the connector 132 and/or the drill 130 such that during use, the guard 110 can remain rotationally constant. The guard 110 may further cover a gap between the aspiration cannula 108 and the connector 132 and/or drill 130, for example, to prevent the operator from pinching his/her hands in the device 100 while the aspiration cannula 108 is rotating.

The distal end of the aspiration cannula 108 can have a tissue disruptor 138, e.g., one or more looped members configured such as a whisk, which may be fixed, coupled, or otherwise integrated with the distal end of the aspiration cannula 108, as described in further detail below. Moreover, the aspiration cannula 108 can facilitate aspiration and/or irrigation by defining one, two, or more lumens therethrough which terminate at corresponding openings at or along a distal portion of the disruptor 138 for aspirating concurrently or subsequently to irrigating.

To provide an initial entry pathway into and through the cortical bone and into the medullary cavity, for instance, an access trocar 134 may be used which has an entry cannula 102 which defines an entry cannula channel that can pass through the length of the access trocar 134. The access trocar 134 can have one or more handles extending laterally and the entry cannula 102 can be configured to drive through cortical bone, for instance using a removable obturator (not shown). Once the trocar 134 has been inserted and desirably positioned within the cortical bone creating an entry point, the aspiration cannula 108 may be passed through the entry cannula channel 102 and into the tissue matrix. If an obturator is used through the trocar 134, the obturator may be removed after insertion through the cortical bone to allow for insertion of aspiration cannula 108 therethrough. Accordingly, channel 102 has a diameter which can reasonably accommodate the outer diameter of aspiration cannula 108.

To direct entry of access trocar 134 and aspiration cannula 108 through the cortical bone and into the bone marrow cavity, a guide 114 can be used to direct one or more surgical devices through a single hole in the tissue. The guide 114 can be used to minimize tissue damage during procedures that otherwise benefit from multiple tool entries through tissue at different angles and/or different adjacent locations. The guide 114 can have a guide body which can be substantially rigid or flexible. The guide body can be made from a polymer, metal, or combinations thereof, and can include a crown which may have a hemi-spherical configuration. A channel or groove defined along guide 114 can be configured to receive or otherwise seat on or adjacent to a target bone to be aspirated and can have a curved or an arcuate configuration formed by a seat wall such as a cylindrical or semi-cylindrical configuration which extends along the length of the guide 114. Within the guide 114, one or more aspiration or guide channels may be defined at predetermined angles such that each of the aspiration channels converge at a single exit port which opens through the seat wall and into the bone seat to consistently direct trocar 134 and/or aspiration cannula 108 through the single puncture opening into the bone marrow cavity at the predetermined angle. Further details and examples of guide 114 and methods for its use are described in U.S. patent application Ser. No. 11/828,048 filed Jul. 25, 2007, which is incorporated herein by reference in its entirety.

The connector and aspiration assembly 132 can have a drill interface 144, such as gearing which engages a complementary interface, which mechanically couples the drill 130 and aspiration assembly 132 to one another via a removable interface which allows the drill interface 144 to couple and de-couple from the drill 130 itself. The drill 130 may be actuated by an actuator control 106 and the connector and aspiration assembly 132 and/or the drill 130 can additionally include a mechanical transmission, for example, to increase and/or decrease the transmitted torque or speed from the drill 130 to the cannula 108. The connector and aspiration assembly 132 and/or the drill 130 can further include a governor regulated by control 112, for example, to limit the rotational speed of the drill 130 transmitted to the aspiration cannula 108. Such a governor can be configured as a resistor, slip-clutch, etc., or combinations thereof. The maximum rotational speed of the aspiration cannula 108 can be from about 30 rpm to about 160 rpm, for example about 120 rpm.

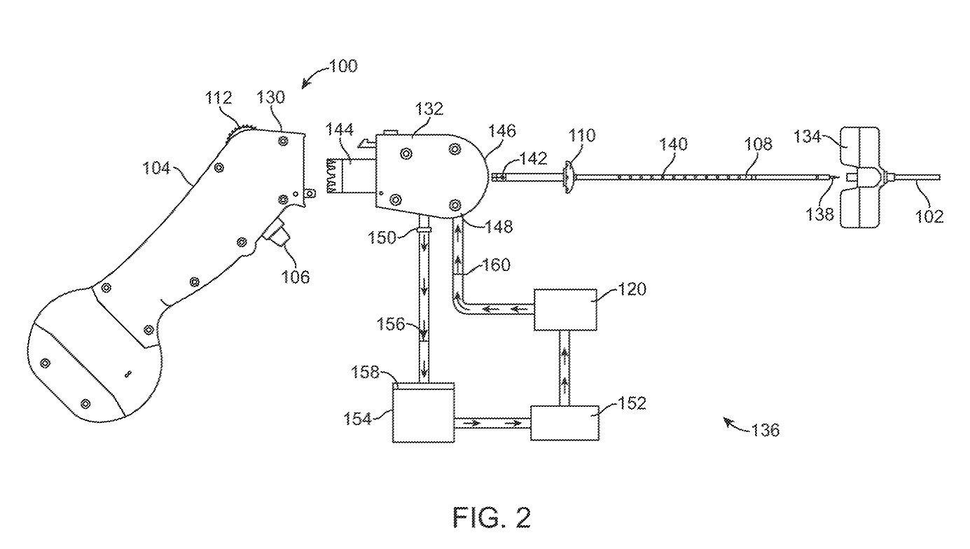

As shown in the schematic assembly view of FIG. 2, connector and aspiration assembly 132 can be further configured to direct and/or control aspiration and/or irrigation between fluid circuit 136 and the first and/or second lumen of the aspiration cannula 108. The connector and aspiration assembly 132 can removably attach to the aspiration cannula 108 at cannula port 146 and the connector and aspiration assembly 132 can further include an irrigation port 148 and/or aspiration port 150, each of which can be configured to be removably attached to fluid lines. The connector and aspiration assembly 132 can be configured to place the irrigation port 148 in fluid communication with a lumen in the aspiration cannula 108, for example a first lumen. The connector and aspiration assembly 132 can be further configured to place the aspiration port 150 in fluid communication with a lumen in the aspiration cannula 108, for example a second lumen, or the same lumen the irrigation port 148 is in fluid communication with.

The fluid circuit 136 can further include a pump 152 which is in fluid communication with an irrigant reservoir 120 and/or an aspirant reservoir 154. The irrigant reservoir 120 can have an irrigant, for example, saline solution. The pump 152 can deliver positive fluid pressure, as shown by arrows, to the irrigant reservoir 120 while also providing negative fluid pressure (i.e., suction), as shown by arrows, to the aspirant reservoir 154. By delivering a positive fluid pressure, the irrigant may be optionally perfused through aspiration cannula 108 into the bone marrow space to facilitate the withdrawal of the disrupted bone marrow. In creating the negative fluid pressure, pump 152 may be accordingly utilized to aspirate the disrupted bone marrow into and through aspiration cannula 108, through aspiration assembly 132, and through aspiration port 150 for deposition into aspirant reservoir 154. The pump 152 can also be configured to reverse direction, i.e., providing negative pressure to the irrigant reservoir 120, and positive fluid pressure to the aspirant reservoir 154, for example, during cleaning to backwash the fluid system or to perfuse fluid into the tissue matrix to facilitate aspiration of the disrupted tissue. In this case, the irrigant perfusion rate can be, for example, from about 1 to 2 cc/min to about 30 cc/min.

An optional first aspiration filter 156 can be positioned in the flow between the aspiration port 150 and the aspirant reservoir 154 while an additional optional second aspiration filter 158 can be positioned in the aspirant reservoir 154, e.g., near the inlet port. An optional irrigation filter 160 can also be positioned between the irrigant reservoir 120 and the irrigation port 148. The first aspiration filter 156 and/or the second aspiration filter 158 can have pore sizes about 10 μm. While filters are shown positioned within the fluid lines or reservoirs, filters may alternatively be positioned within the cannula 108 itself, e.g., near or at the distal tip, for filtering out undesirable debris during aspiration such that the debris is prevented from passing through the cannula 108 and/or connector and aspiration assembly 132.

The drill 130, having a handle 104 and controls 106, 112, can include any number of drills which are available for surgical purposes as interface 144 may be configured with a standard interface to couple and de-couple from any conventional drill interface. Examples of such drills 130 may include, for example, drills from DePuy Mitek, Inc. (Raynham, Mass.), Aesculap, Inc. (Center Valley, Pa.), Universal Driver or C.O.R.E. Micro Drill, Impaction Drill, Universal Series Drill (e.g., UHT Drill, U Drill), or Saber Drill commercially available from Stryker Corp. (Kalamazoo, Mich.), etc.

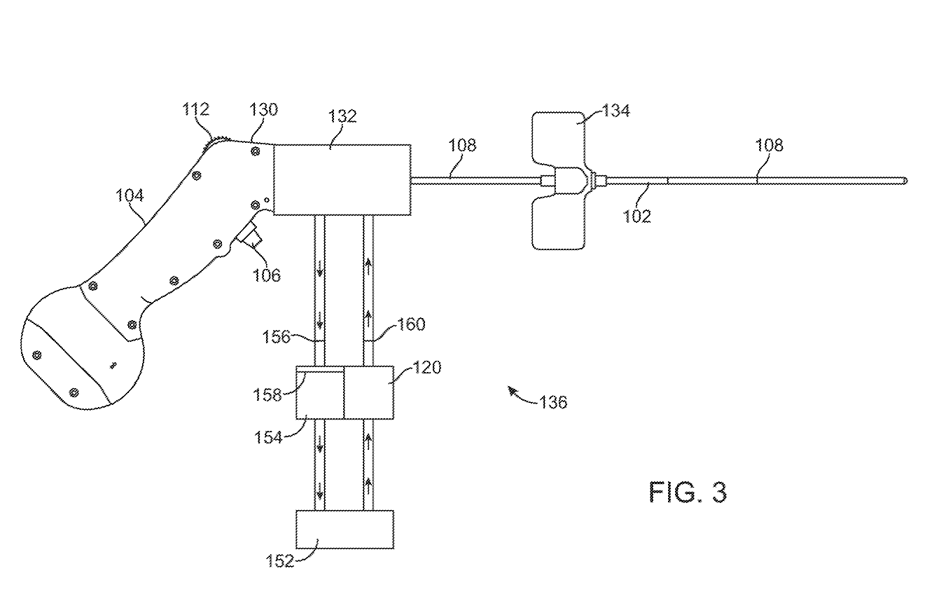

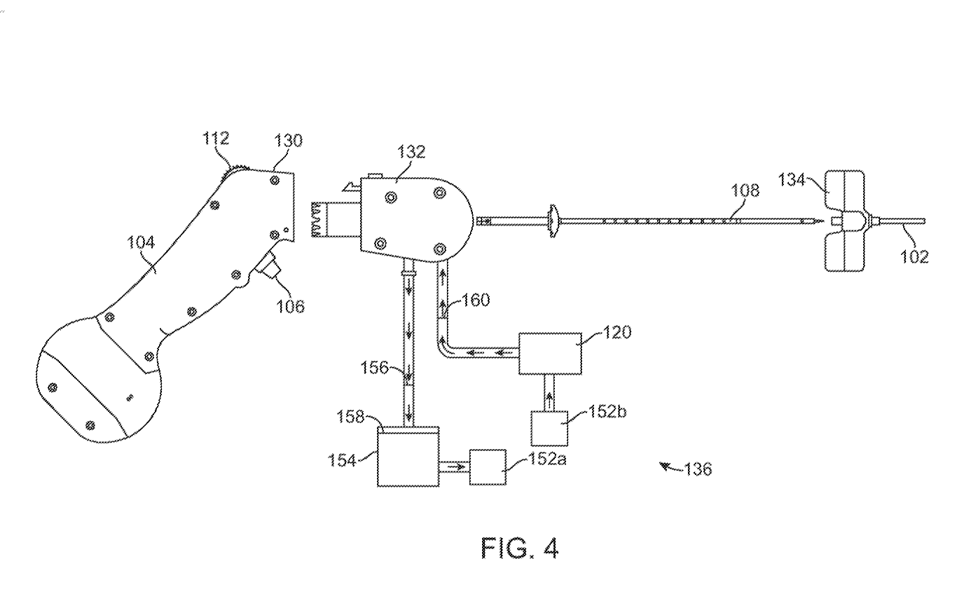

FIG. 3 illustrates another variation showing the aspirant reservoir 154 and the irrigant reservoir 120 integrated and/or attached to one another. As further shown, drill 130 is engaged to connector and aspiration assembly 132. FIG. 4 illustrates yet another variation where the fluid circuit 136 can have separated irrigation and aspiration fluid flow sub-circuits. The irrigation sub-circuit can have an irrigation pump 152 while the aspiration sub-circuit can have an aspiration pump 152a separated from the irrigation pump 152b.

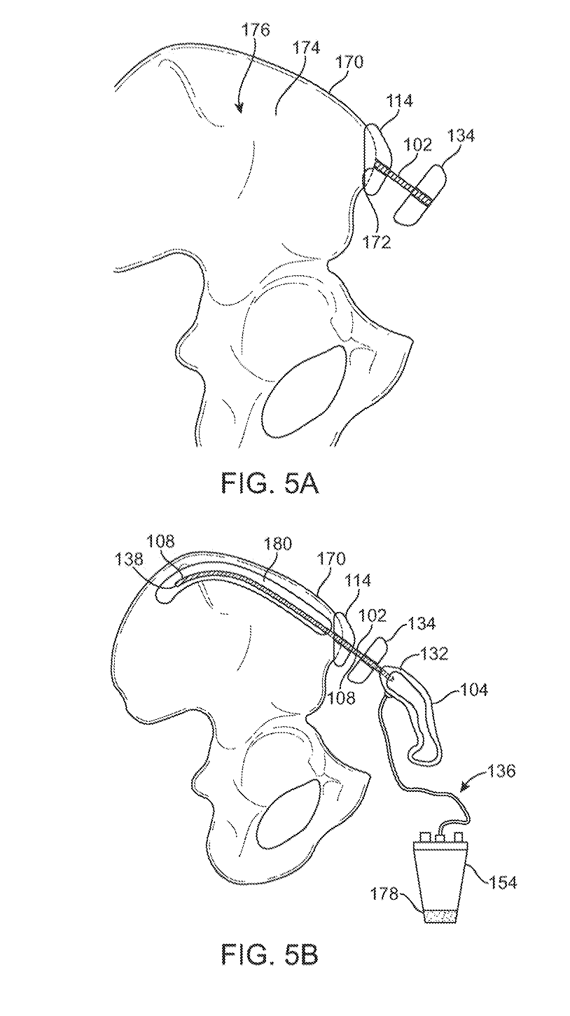

In use, one method is illustrated in FIGS. 5A to 5D which show guide 114 placed upon the patient's skin over an entry target site 172 such as an anterior portion along the crest of the iliac 170. Access trocar 134 may be advanced through an entry passage of guide 114, which directs the trocar 134 at a desired angle relative to the target site 172, such that trocar 134 is inserted percutaneously through the patient's skin and into the target site 172. Access trocar 134 may pierce at least partially into the intramedullary bone marrow space 176 of the iliac 170 such that entry cannula 102 provides a direct access route to the bone marrow 174 residing within space 176, as shown in FIG. 5A.

Aspiration cannula 108 may then be advanced through trocar 134 and entry cannula 102 into the space 176 along the interior of the crest of the iliac 170 where cannula 108 may be activated to rotate tissue disruptor 138 to disrupt the bone marrow 174 from the surrounding tissue matrix. As shown in this example, cannula 108 may be advanced from an anterior position to a posterior position within the space 176 although in other methods of use, cannula 108 may be inserted through a posterior location along iliac 170 such that cannula 108 is advanced from a posterior position to an anterior position within space 176. As tissue disruptor 138 is rotated, it may be advanced distally to follow along the crest of the iliac 170 through the bone marrow 174 while aspirating the disrupted bone marrow 174. Optionally, aspiration cannula 108 may be advanced distally until fully disposed through space 176 where the disrupted tissue may be aspirated while cannula 108 is withdrawn proximally relative to iliac 170. Moreover, a fluid such as saline may be infused through cannula 108 and into the disrupted bone marrow 174 while cannula 108 is advanced distally and/or withdrawn proximally to facilitate aspiration of the tissue.

As illustrated in FIG. 5B, as the disrupted bone marrow 174 is aspirated through aspiration cannula 108, the aspirant is withdrawn through aspiration assembly 132 and fluid circuit 136 where aspirated bone marrow 178 may be collected within reservoir 154. A channel, e.g., first aspirated channel 180, may be formed through the bone marrow 174 within iliac 170 where the bone marrow has been aspirated through cannula 108. Aspiration cannula 108 may then be withdrawn from access trocar 134 and reinserted or readjusted through another opening within guide 114 such that cannula 108 is advanced into the same entry port through iliac 170 but at a different angle. The adjustment and reposition can be concurrent with rotation of the aspiration cannula 108, for example, to disrupt additional bone marrow or the adjustment and repositioning can occur without rotating the aspiration cannula 108.

As shown in FIG. 5C, with aspiration cannula 108 readjusted and reinserted into iliac 170, the bone marrow 174 may be aspirated along a second path adjacent to the first aspirated channel 180. The aspirated tissue may be withdrawn from iliac 170 and collected in reservoir 178 leaving a channel, e.g., second aspirated channel 182, defined through the bone marrow 174 and adjacent to first aspirated channel 180. Aspiration cannula 108 may again be withdrawn from trocar 134 and guide 114 and repositioned to enter through guide 114 and through the same opening at yet another angle relative to iliac 170. As illustrated in FIG. 5D, aspiration cannula 108 may again be advanced from an anterior to posterior position within space 176 while directing tissue disruptor 138 and aspiration cannula 108 inferiorly relative to the puncture opening along a third path. The aspirated tissue may leave a third aspirated channel 184 within the bone marrow 174.

Although three aspiration paths are illustrated in this example, fewer or more than three paths may be taken depending upon the desired amount of bone marrow to be harvested. In one example, the aspiration cannula 108 may be utilized to obtain between 20 to 200 ml of bone marrow volume per pass through the space 176 and preferably about 40 ml of bone marrow volume per pass. Furthermore, aspiration cannula 108 may be utilized to collectively obtain between 200 to 300 ml of bone marrow volume per procedure through a single opening along the iliac crest.

Additional examples and details of methods and devices which may be utilized with the systems described are shown in U.S. patent application Ser. Nos. 10/454,846 filed Jun. 4, 2003 and 11/750,287 filed May 17, 2007, each of which is incorporated herein by reference in its entirety.

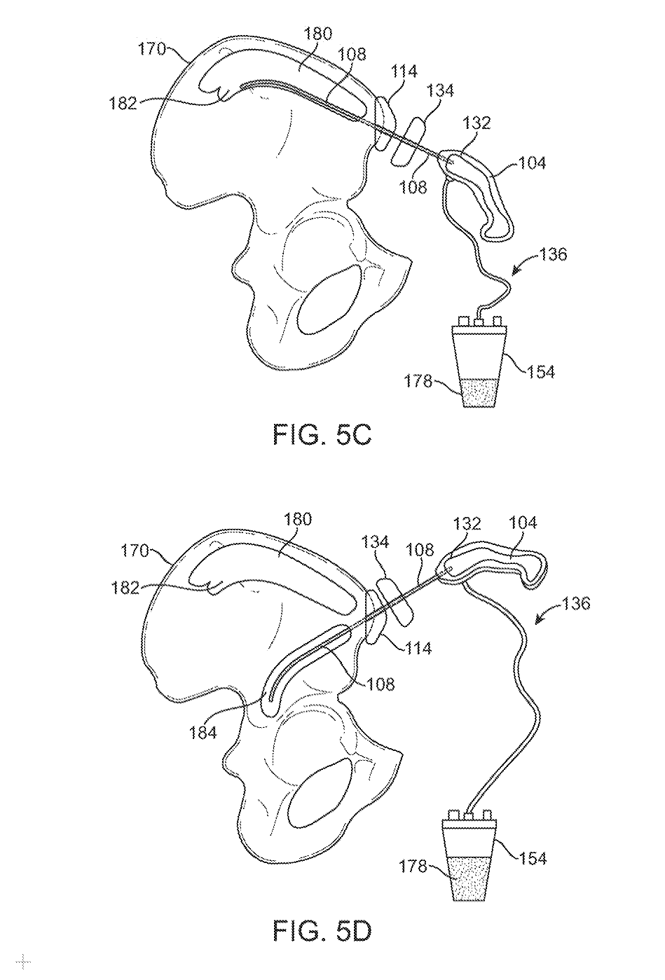

Turning now to the handle, FIG. 6 shows a cross-sectional perspective view of one variation of handle 104. Handle 104 may be configured to actuate and rotate aspiration cannula 108 via motor 190 which is driven by power supply 192, e.g., a battery or rechargeable battery, and activated via actuator control 106. A mechanical transmission 194 may be coupled to motor 190 to limit or control the rotational speed of motor 194 depending upon the actuation of control 112 to either increase, decrease, or limit the speed at which motor 190 rotates aspiration cannula 108. Mating gear 196 may be coupled to transmission 194, or directly to motor 190, for engagement with drill interface 144 extending rotatably from aspiration assembly 132. Rotational interface 142, which extends from the proximal end of aspiration cannula 108, may be coupled to drill interface 144 such that rotation of mating gear 196 transfers rotational torque to rotational interface 142 via drill interlace 144 to rotate aspiration cannula 108 about its longitudinal axis.

Aspiration assembly 132 is removably coupled to handle 104 and may be secured to handle 104 via a locking mechanism 198, which may be releasable via lock release 200. Aspiration cannula 108 may be inserted into assembly 132 before or after assembly 132 is securely coupled to handle 104 and secured via rotational interface 142 and/or guard 110, which may also limit the advancement of cannula 108 into assembly 132. The proximal end of aspiration cannula 108 inserted into assembly 132 may generally comprise an aspiration assembly interface 202 extending proximally from the shaft of cannula 108 and terminating with rotational interface 142.

A plurality of openings 204 may be defined along aspiration assembly interface 202 such that bone marrow and/or other aspirants which are drawn proximally through cannula 108 may enter aspiration assembly interface 202 to exit through openings 204 and into aspirant chamber 206, which is defined by a cavity contained within assembly 132. Seals or gaskets 208 may be positioned at proximal and distal ends of chamber 206 such that the insertion of aspiration assembly interface 202 within assembly 132 positions openings 204 within chamber 206 between seals 208. Moreover, as aspiration assembly interface 202 is rotated within assembly 132 and relative to seals 208, the outer surface of aspiration assembly interface 202 may maintain its fluid-tight interface with respect to seals 208 such that aspirants and fluids are contained within chamber 206. As cannula 108 and aspiration assembly interface 202 are rotated, the bone marrow and/or aspirant drawn through openings 204 and collected within chamber 206 may be removed from assembly 132 via aspirant port opening 210, which is in fluid communication with fluid circuit 136, as described above. This particular variation is intended to be illustrative of some of the mechanisms which may be utilized for aspirating bone marrow and/or other aspirants. Accordingly, other mechanisms and systems which may be utilized with or within the handle 104 are intended to be included in this description.

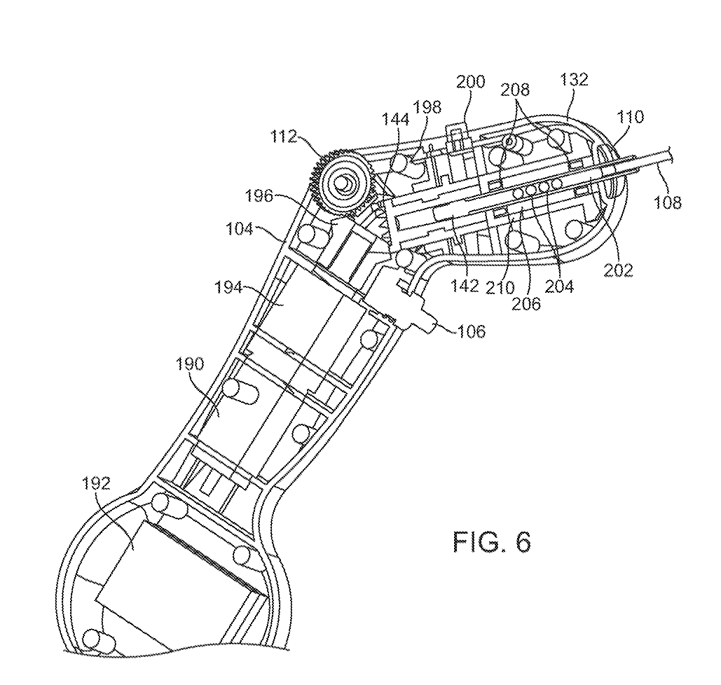

Turning now to aspiration cannula 108, as shown in the side view of FIG. 7A, another variation of aspiration assembly interface 220 is illustrated having an opening 204 configured as an elongate slot proximal to guard 110. Cannula 108 may be coupled or secured to interface 220, as shown in the cross-sectional detail side view of FIG. 7B, by a number of mechanisms. In this variation, a proximal portion of cannula 108 may be inserted partially within and secured to interface 220. The shaft of cannula 108 extends distally and may generally comprise a proximal portion 222 and a distal portion 226 with a transition portion 224 therebetween. Although cannula 108 may generally be flexible enough to allow for bending or curvature of the shaft when advanced within and/or against the bone cavity interior, cannula 108 is desirably stiff enough to transmit between 20 to 40 in·oz, and preferably 40 in·oz, of torque to rotate cannula 108 through the bone marrow. While cannula 108 may have an overall length sufficient for the device to be advanced throughout the bone cavity (e.g., about 9.45 inches) proximal portion 222 may extend anywhere from ½ to ⅔ of the length of cannula 108 (e.g., 6 to 6.7 inches) while distal portion may extend anywhere from ⅓ to ½ of the length of cannula 108 (e.g., 2.6 to 2.7 inches) in a manner complementary to the proximal portion 222. A transition portion 224 between the proximal and distal portions 222, 226 may have a length ranging from, e.g., 0.1 to 1.1 inches.

As described above, the distal portion of cannula 108 may comprise tissue disruptor assembly 138 which may be configured in a number of different variations. One variation is illustrated in the partial cross-sectional detail view of FIG. 7C which shows tissue disruptor 234, e.g., looped member such as a looped wire, retained within disruptor tube member 230, which also defines one or more aspiration ports 232 proximal to disrupter 234 along a side surface of disruptor tube member 230. A proximal portion of tube member 230 may be retained within a distal end of distal portion 226 and secured via a crimped member or swage tube 228 disposed over and securing both the cannula shaft and tube member 230.

As also described above, cannula 108 may define one or more visualization markers 140 along a portion or an entire length of its outer surface at regular intervals and/or at preset distances to provide a visual indication to the user of a depth of aspiration cannula 108 within the body cavity.

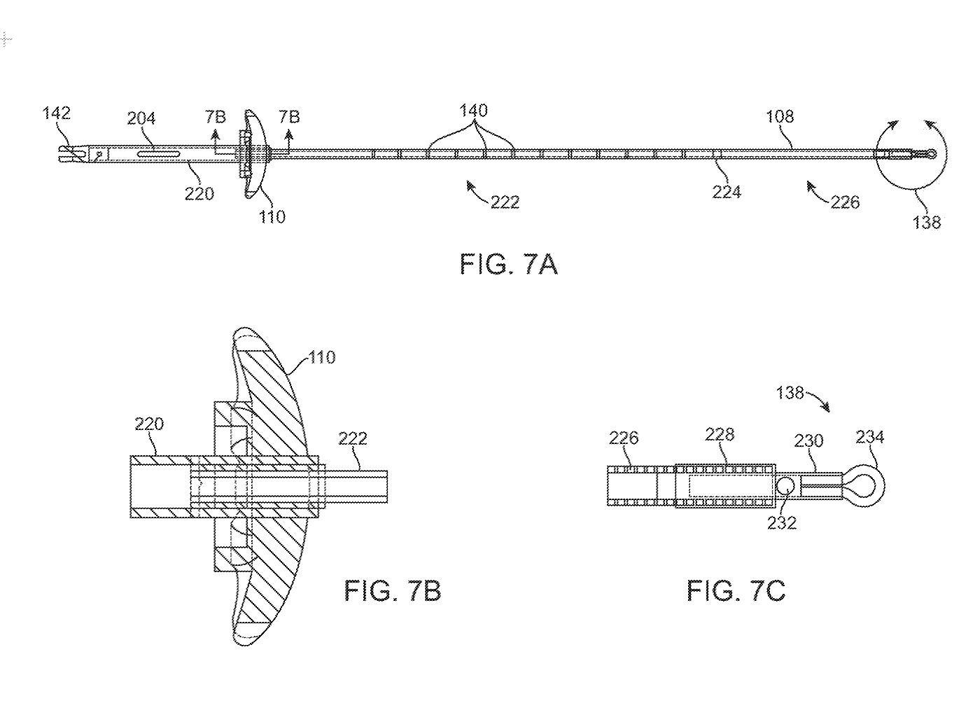

Although the proximal portion 222 of cannula 108 may generally be stiffer relative to distal portion 226, the aspiration lumen defined through the length of cannula 108 may remain relatively constant. For instance, the internal diameter of cannula 108 may be based upon the standard dimensions of a 12 gauge needle, e.g., 0.085 inch, or any other suitable non-standard diameter. FIG. 8B illustrates a representative cross-sectional end view of cannula 108 along the proximal portion 222 where multiple layers of material may be overlaid to create the desired stiffness along the proximal portion 222. In this particular variation, a first polyimide layer 240 having a wall thickness ranging from 0.001 to 0.010 inch, e.g., 0.005 inch, may form the aspiration lumen. An additional second polyimide layer 242 also having an exemplary wall thickness ranging from 0.001 to 0.010 inch, e.g., 0.005 inch, may be overlaid upon polyimide layer 240 to provide additional stiffness to the proximal portion 222 of cannula 108. This second polyimide layer 242 may extend along the length of proximal portion 222 and terminate proximal to, at, or along the transition portion 224 of cannula 108.

As further illustrated, a first braid layer 244 may be overlaid atop second polyimide layer 242 to provide for improved torsional transmission while retaining flexibility along proximal portion 222. Such a braid layer 244 may be fabricated from a number of various materials and at various braid pitch angles although this particular variation illustrates a stainless steel braid fabricated from wire or ribbon having a 0.0015 inch×0.0090 inch dimension. The braid pitch may be varied although in this example, the proximal portion of braid layer 244 may be configured at 25 threads per inch (TPI). Atop the first braid layer 244, first nylon layer 246, e.g., VESTAMID® L21101 NYLON (Degussa-Huls Aktiengesellschaft Corp., Germany), may be overlaid and atop first nylon layer 246, a second braid layer 248 having the same (or different) characteristics as first braid layer 244 may be overlaid. Although nylon is an exemplary material, any number of other relatively high-Durometer polymers may also be utilized, e.g., polyurethane, PEBAX® (Arkema France Corp., Puteaux, France), etc.

Second braid layer 248 is optional and may be omitted entirely from the shaft depending upon the desired strength and torque capabilities. Finally, a second nylon layer 250 may be overlaid upon second braid layer 248, if present. Alternatively, if second braid layer 248 is omitted entirely, first and/or second nylon layer 246, 250 may be overlaid directly upon the first braid layer 244. The multiple layering of materials may combine to form a proximal portion 222 having an outer diameter of, e.g., 0.128 inches, and a shaft which is sufficiently flexible yet rigid enough to transmit the desired torque along the length of cannula 108.

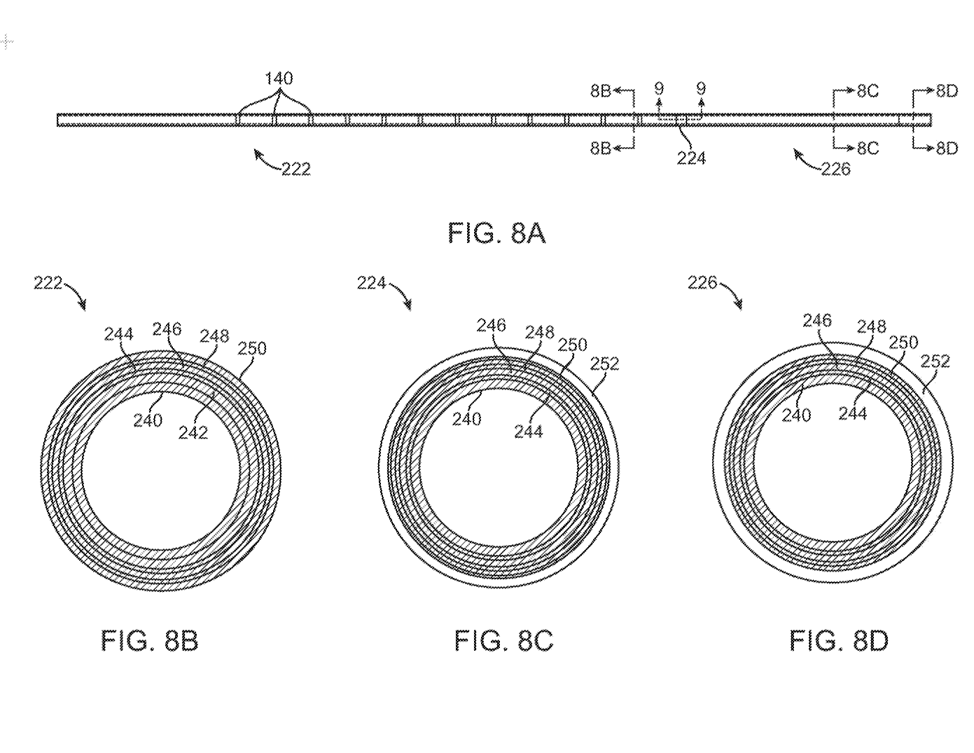

FIG. 8C illustrates a cross-sectional end view of transition portion 224, which shows the outer surface 252 of cannula 108 tapering down from an outer diameter of, e.g., 0.128 inches, along proximal portion 222 to an outer diameter of, e.g., 0.118 inches, along distal portion 226. Also, second polyimide layer 242 may end proximal to, at, or along the transition portion 224 while the remaining layers continue to extend along cannula 108. Moreover, one or both braid layers 244, 248 may transition from 25 TPI along proximal portion 222 to 45 TPI along distal portion 226 over transition portion 224. FIG. 8D illustrates the cross-sectional end view of distal portion 226, which shows one or both braid layers 244, 248 with, e.g., 45 TPI, and a reduced outer diameter of 0.118 inches. FIG. 9 illustrates a cross-sectional side view of the transition portion 224 from FIG. 8A. As shown in this variation, second polyimide layer 242 terminates at the beginning of transition portion 224 and the braid pitch along one or both braid layers 244, 248 may transition from 25 TPI along proximal portion 222 to 45 TPI along distal portion 226.

Generally, because the shaft itself is rotating about its longitudinal axis while providing an aspiration lumen, the shaft transmits a torque along its length rather than through a separate drive shaft. Accordingly, the combination of the various layers provides a balance which results in the desired strength, flexibility, and torque transmission characteristics for a shaft which is suitable to be introduced into the iliac crest to flexibly advance through the bone cavity while rotating to aspirate disrupted tissue therethrough. These examples are intended to be illustrative of variations for overlaying various layers upon one another to attain an aspiration cannula 108 having the desired stiffness and bending characteristics along its length. Other variations for attaining the desired stiffness by altering braid pitch or layer characteristics may be utilized.

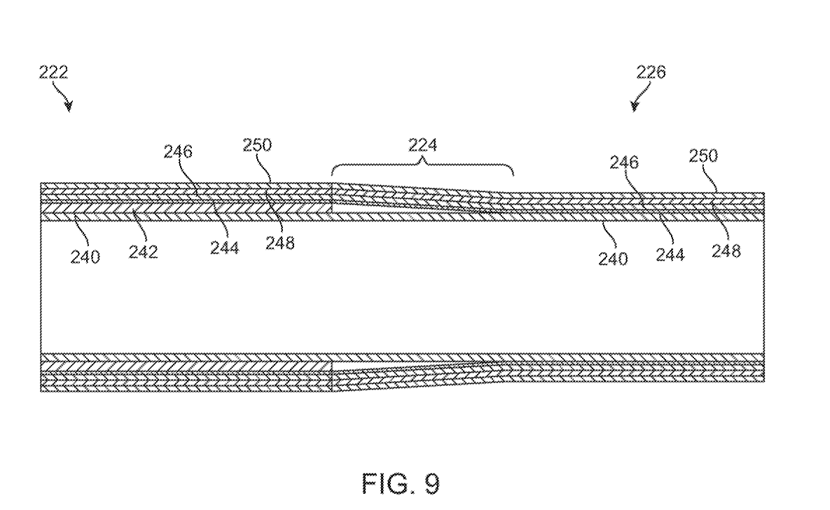

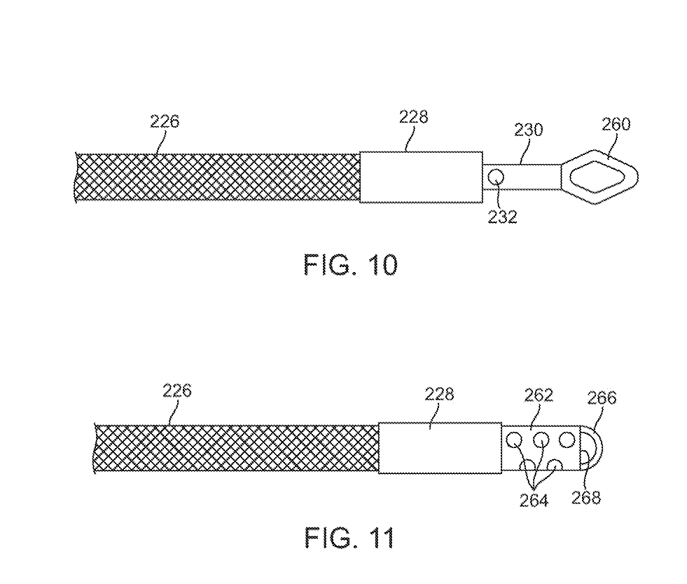

Turning now to additional variations for the tissue disruptor, FIG. 10 shows a detail side view of another variation where a looped disruptor 260 which is tapered may be retained in disrupter tube member 230. As described above in FIG. 7C, looped disruptor 260 may be retained in swage tube 230 which may also define one or more aspiration openings 232 along a side surface of tube 230. Aspiration openings 232 in this and other variations may be defined along a side surface of the tube 230 or along an outer side surface of aspiration cannula 226 to prevent clogging of the openings 232 by bone marrow or other aspirants during an aspiration procedure. FIG. 11 shows yet another variation where looped tissue disruptor 266 may extend from swage tube 262, which may define one or more aspiration openings 264 along the side surfaces of tube 262. A distal end of swage tube 262 proximal to looped tissue disruptor 264 may incorporate an occluded distal end 268 (e.g., occluded with solder) to prevent bone marrow or aspirants from entering through an opening over the distal tip and potentially clogging or blocking the cannula 108.

The looped tissue disrupter may be configured to be advanced within and to disrupt the tissue matrix and bone marrow within the body space while rotated. However, the looped distal end is desirably atraumatic such that piercing through the surrounding cortical bone is inhibited or prevented. Thus, when the looped tissue disruptor is advanced against the walls of the bone space, the disruptor may be deflected to slide or follow along the bone surface rather than piercing through the bone wall.

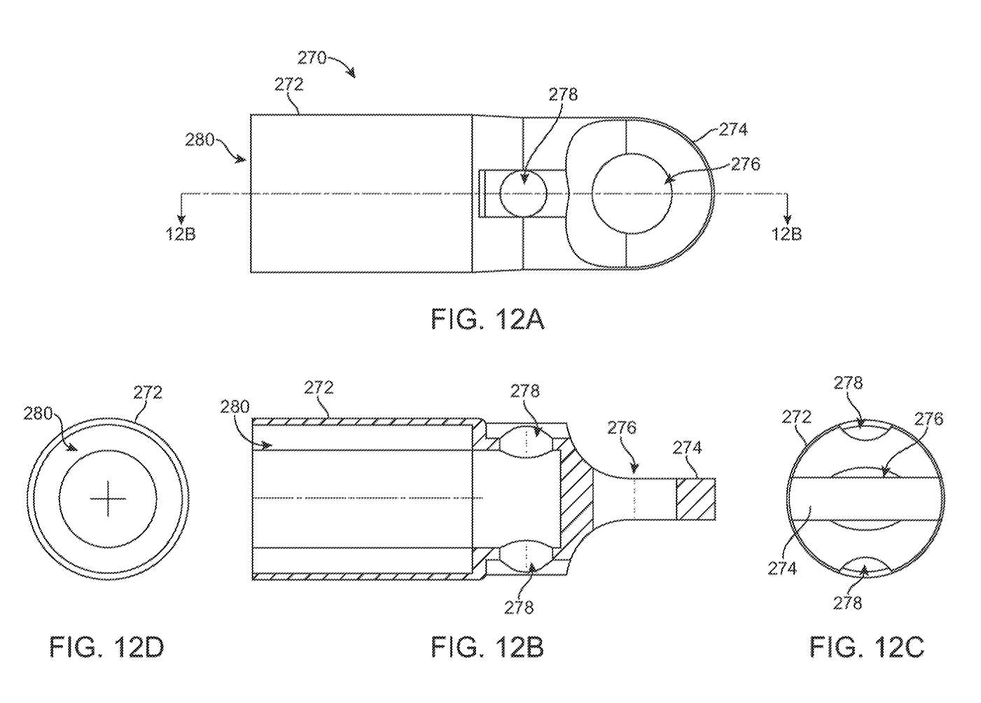

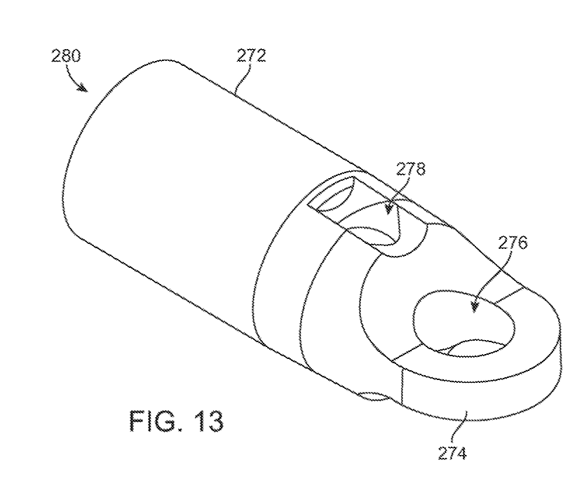

In yet another variation of the tissue disruptor, FIG. 12A shows a side view of a unitary disruptor tip which may be swaged or otherwise attached to the distal end of a cannula shaft. Unitary tissue disrupter 270 may generally comprise a curved or semicircular disruptor member 274 which extends distally from tubular member 272 to form an opening 276. One or more aspiration openings 278 may be defined along tubular member 272 proximal to disruptor member 274 such that the aspiration openings 278 are in communication with lumen 280 defined through tubular member 272, as shown in the cross-sectional side view of FIG. 12B. The portion of tubular member 272 proximal to disrupter member 274 may be occluded such that the only aspiration openings 278 are located along the side surfaces of tubular member 272 to provide for aspiration therethrough, as shown in the respective end views of FIGS. 12D and 12C. FIG. 13 shows a perspective view of the tissue disrupter illustrating the integral nature of the tissue disruptor as well as the positioning of the aspiration openings 278 proximally of disrupter member 274 and opening 276.

Such a unitary tissue disruptor may be fabricated as a single and integral unit, e.g., from stainless steel or any other suitable material. Tissue disrupter 270 may also be sized suitably for coupling to the distal end of the cannula shaft and for insertion into the bone cavity. Accordingly, tissue disruptor 270 may have a length of, e.g., 0.369 inches, with an outer diameter of, e.g., 0.130 inches. Moreover, tubular body 272 may be stiff enough to provide for a relatively thin wall of, e.g., 0.005 inches, such that the inner diameter of lumen 280 is sufficiently large, e.g., 0.120 inches, to accommodate the aspiration of bone marrow and/or other aspirants therethrough. Moreover, disrupter member 274 may be sufficiently sized to have an opening 276, e.g., 0.067 inches, which is large enough to disrupt the tissue matrix when rotated within the bone cavity.

When utilizing the devices above to aspirate along a path through the bone marrow within the iliac 170, a void or channel may be created (at least temporarily) within the bone marrow where the aspirated tissue has been removed. If aspiration cannula 108 is then withdrawn, repositioned, and reintroduced into the bone cavity along a second path which is adjacent to the first aspirated channel 180, then aspiration cannula 108 may inadvertently cross one or more times into the emptied first aspirated channel 180. To inhibit or prevent this from occurring, a space-occupying member 290 may be inserted through the puncture opening and into the first aspirated channel 180 to temporarily occupy the emptied volume. Space-occupying member 290 may have a length which approximates that of the aspiration cannula 108 such that most, if not all, of the empty space within the aspirated channel is occupied.

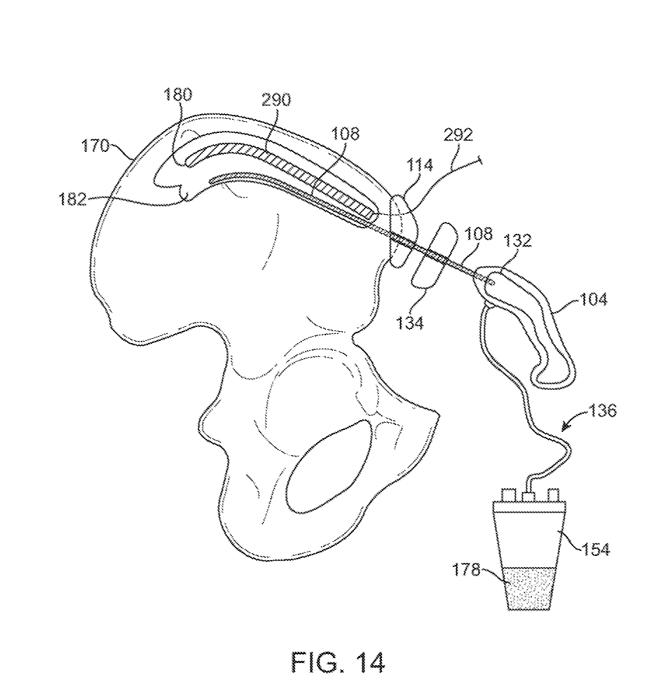

With member 290 occupying the emptied channel, reinsertion and re-advancement of aspiration cannula 108 along an adjacent path may be accomplished while inhibiting or preventing cannula 108 from crossing into the emptied space by member 290. Thus, additional bone marrow may be aspirated along second aspirated channel 182 adjacent to first aspirated channel 180, as illustrated in FIG. 14.

Space-occupying member 290 may be comprised of various biocompatible materials and is sufficiently sized and flexible to be inserted and placed within the emptied bone marrow channel. Accordingly, member 290 may be fabricated from a variety of polymers or plastics. A wire 292 may be attached to a proximal end of member 290 to allow for removal of the member 290 upon completion of the bone marrow harvesting procedure. Alternatively, member 290 may be fabricated from a bioabsorbable or biodegradable polymer which may be left within the iliac 170 to become absorbed or simply implanted in place.

In yet another alternative, a variation of cannula shaft 108 may be detached from aspiration assembly 132 and cannula 108 may be left in place within first aspirated channel 180 to occupy the space. A second aspiration cannula may be attached to assembly 132 and reinserted for advancement along the second adjacent path such that the detached aspiration cannula 108 functions as the space-occupying member. Upon completion of the procedure, both the second and the first cannula 108 may be removed from the patient body.

It is apparent to one skilled in the art that various changes and modifications can be made to this disclosure, and equivalents employed, without departing from the spirit and scope of the invention. Elements shown with any variation are exemplary for the specific variation and can be used on or in combination with any other variation within this disclosure.

We claim:

1. An apparatus for tissue aspiration, comprising:
an elongated cannula having a flexible length and at least one lumen defined therethrough;
a tissue disruptor positioned upon a distal end of the cannula and having a looped member with at least one aspiration opening defined along a side surface proximal of the looped member in communication with the lumen,
wherein the disruptor is configured to rotate about an axis while moved longitudinally through tissue such that 20 to 200 ml of disrupted tissue is aspirated through the at least one aspiration opening per pass through the tissue.

2. The apparatus of claim 1 wherein the elongated cannula comprises a proximal portion, a transition portion, and a distal portion wherein the proximal portion is relatively stiffer than the distal portion.

3. The apparatus of claim 2 wherein the cannula is configured to transmit 20 to 40 in·oz of torque along a length of the cannula.

4. The apparatus of claim 2 wherein the proximal portion comprises at least two layers of polyimide.

5. The apparatus of claim 2 wherein the cannula comprises at least two layers of nylon.

6. The apparatus of clam 2 wherein the cannula comprises at least one braid layer having a first stiffness along the proximal portion and a second stiffness along the distal portion.

7. The apparatus of claim 2 wherein the proximal portion of the cannula has a first diameter and the distal portion of the cannula has a second diameter, which is less than the first diameter.

8. The apparatus of claim 1 wherein the tissue disruptor comprises a unitary member having an occluded distal tip.

9. The apparatus of claim 1 further comprising an aspiration assembly configured to rotatingly receive a proximal end of the elongated cannula.

10. The apparatus of claim 9 wherein the aspiration assembly defines an aspiration chamber within which receives aspirated tissue from the cannula while the cannula is rotated with respect to the chamber.

11. The apparatus of claim 1 further comprising an access guide configured to position the elongated cannula at a predetermined angle relative to the tissue.

12. The apparatus of claim 1 wherein the elongated cannula comprises at least one internal layer of polyimide, at least one braided layer overlaid atop the layer of polyimide, and at least one layer of a high-durometer polymer overlaid atop the braided layer.

13. A method for removing bone marrow from a subject, comprising:
advancing a distal end of an elongated cannula having a flexible length with a tissue disrupter attached to a distal end thereof into a body cavity of a patient through a single opening in the cavity;
rotating the tissue disruptor while advancing the cannula along a first path such that a tissue matrix within the body cavity is disrupted; and
aspirating 20 to 200 ml of the disrupted tissue matrix per pass through the tissue matrix via at least one aspiration opening defined along a side surface of the tissue disruptor.

14. The method of claim 13 wherein advancing comprises introducing the cannula through the single opening into an iliac crest.

15. The method of claim 13 wherein advancing further comprises directing the cannula through the single opening at a predetermined angle via an access guide.

16. The method of claim 13 wherein the cannula comprises a proximal portion, a transition portion, and a distal portion wherein the proximal portion is relatively stiffer than the distal portion.

17. The method of claim 13 wherein rotating comprises transmitting 20 to 40 in-oz of torque along a length of the cannula.

18. The method of claim 13 wherein rotating comprises rotating a looped member extending from the tissue disruptor within the tissue matrix.

19. The method of claim 13 wherein aspirating comprises aspirating the disrupted tissue matrix while rotating the tissue disruptor.

20. The method of claim 13 wherein aspirating comprises collecting the disrupted tissue matrix within an aspiration chamber while rotating the cannula with respect to the chamber.

21. The method of claim 13 further comprising perfusing the tissue matrix with a fluid prior to aspirating.

22. The method of claim 13 further comprising withdrawing the cannula from the opening in the cavity.

23. The method of claim 22 further comprising inserting a space-occupying member within a tissue channel formed by aspirated tissue.

24. The method of claim 23 further comprising reintroducing the cannula through the opening in the cavity at a second angle.

25. The method of claim 24 further comprising re-aspirating 20 to 200 ml of disrupted tissue along a second path adjacent to the first path and the space-occupying member.

26. A tissue disruptor apparatus, comprising:
a tubular member defining a lumen therethrough;
a looped member projecting distally from the tubular member and defining an opening through the looped member such that the looped member is integrally formed with the tubular member;
one or more aspiration openings defined along a side surface of the tubular member proximal to the looped member, wherein the one or more aspiration openings are in fluid communication with the lumen, and
wherein the tubular member proximal to the looped member is occluded.

27. The apparatus of claim 26 wherein the one or more aspiration openings are sized to aspirate 20 to 200 ml of disrupted tissue per pass through the tissue.

28. The apparatus of claim 26 further comprising an elongate cannula coupled to the tissue disrupter.

29. An elongate flexible shaft defining an aspiration lumen therethrough, the shaft comprising:
a first layer of polyimide defining an inner diameter consistent through a length of the lumen;
a first braided layer overlaid atop the first layer of polyimide; and
a first layer of high-durometer polymer overlaid atop the first braided layer, wherein a proximal portion of the shaft is stiffer relative to a distal portion of the shaft.

30. The shaft of claim 29 further comprising a second layer of polyimide between the first layer of polyimide and the first braided layer along the proximal portion of the shaft.

31. The shaft of claim 29 wherein the first layer of polyamide has a thickness of between 0.001 to 0.010 inch.

32. The shaft of claim 29 wherein the first braided layer comprises a stainless steel braid defining 25 threads per inch along the proximal portion of the shaft and 45 threads per inch along the distal portion of the shaft.

33. The shaft of claim 29 wherein the first layer of high-durometer polymer comprises nylon.

34. The shaft of claim 29 further comprising a second braided layer overlaid atop the first layer of high-durometer polymer.

35. The shaft of claim 34 further comprising a second high-durometer polymer overlaid atop the second braided layer.

36. The shaft of claim 29 wherein the inner diameter is 0.085 inch along the length of the lumen.

37. The shaft of claim 29 wherein an outer diameter of the shaft along the proximal portion is greater than an outer diameter of the shaft along the distal portion.

38. The shaft of claim 37 wherein the outer diameter is 0.128 inch along the proximal portion and 0.118 inch along the distal portion of the shaft.

39. The shaft of claim 29 further comprising a tissue disrupter positioned upon a distal end of the shaft and having a looped member with at least one aspiration opening defined along a side surface proximal of the looped member in communication with the lumen.

40. The shaft of claim 39 wherein the tissue disruptor is configured to rotate about an axis while moved longitudinally through tissue such that 20 to 200 ml of disrupted tissue is aspirated through the at least one aspiration opening per pass through the tissue.
